(19) United States
(12) Patent Application Publication
(10) Pub. No.: US 2010/0202983 A1
(43) Pub. Date: Aug. 12, 2010

(54) SELECTIVELY TARGETED ANTIMICROBIALS FOR THE TREATMENT OF PERIODONTAL DISEASE

(76) Inventor: Gary R. Jernberg, North Mankato, MN (US)

Correspondence Address: MERCHANT & GOULD PC, P.O. BOX 2903, MINNEAPOLIS, MN, 55402-0903, US

(21) Appl. No.: 12/700,396

(22) Filed: Feb. 4, 2010

Related U.S. Application Data
(60) Provisional application No. 61/150,865, filed on Feb. 9, 2009.

(51) Int. Cl. A61K 8/72; A61K 38/16; A61P 43/00; A61Q 11/00

(52) U.S. Cl. 424/49; 514/2

(57) ABSTRACT

Compositions and methods for local delivery of selectively targeted antimicrobials are disclosed for the treatment of periodontal disease by eliminating or significantly reducing specific pathogenic bacteria from the bacterial biofilm (plaque) occupying the gingival crevice or periodontal pocket. A carrier agent is utilized to allow for retention, penetration and sustained release.

### CROSS REFERENCE TO RELATED APPLICATIONS

This application claims benefit of Provisional Application No. 61/150,865, filed Feb. 9, 2009, which application is incorporated herein by reference.

### TECHNICAL FIELD

The present invention relates to compositions and methods of treating periodontal disease and related disorders utilizing a sustained, controlled release of selectively targeted antimicrobials to eliminate or significantly reduce specific bacterial pathogens from the periodontal biofilm.

### BACKGROUND OF THE INVENTION

Periodontal diseases are a major dental affliction and health concern to mankind. Gingivitis, inflammation of gingival (gum) tissue, and periodontitis, inflammation and progressive loss of ligament and alveolar (socket) bone support to teeth, are caused by specific bacteria which colonize and occupy the gingival crevice region as part of multispecies bacterial biofilms (plaque). Out of the approximately seven hundred bacterial species found in the oral cavity, a relatively small number of species are implicated in the etiology of periodontal disease.

Although specific bacteria are essential agents for periodontal disease, their presence alone in the periodontal crevice or pocket is not sufficient to explain the periodontal disease process. The host must react to this bacterial challenge if disease is to develop and progress. As with other bacterial infections, the host's immune system acts locally at the invasion site and attempts rapidly to neutralize, remove or destroy the bacterial agents. In periodontal disease, however, chronic bacterial plaque accumulation causes an excessive and persistent antigenic stimulus. Therefore, the host response, rather than being protective and self-limiting, can be destructive. See R. C. Page, Periodontal Disease, p. 221, Lea and Febiger, Philadelphia, 1989.

Periodontal disease is characterized by a chronic inflammatory lesion and the lack of normal resolution. The chronic inflammatory state may facilitate the persistence of periodontal pathogens within the plaque. See H. Hasturk, et al, Resolvin E1 regulates inflammation at the cellular and tissue level and restores tissue homeostasis in vivo, J. Immunology, 179:7021-7029 (2007).

In early stages a biofilm is comprised of a cell layer attached to a surface. The cells grow and divide, forming a dense mat numerous layers thick. When sufficient numbers of bacteria are present (quorum) they signal each other to reorganize forming an array of pillars and irregular surface structures, all connected by convoluted channels that deliver food and remove waste. The biofilm produces an extracellular (glycocalyx) matrix shielding the bacteria from the environment.

As the biofilm matures, the bacteria become greatly more resistant to antibiotics than when in the planktonic (free cell) state. See H. Anwar, et al, Establishment of aging biofilms: a possible mechanism of bacterial resistance to antimicrobial therapy, Antimicrob Agents Chemother 36:1347 (1992). The host immune system is also significantly less effective against bacteria in the biofilm state. See E. T. Jensen, et al, Human polymorphonuclear leukocyte response to Pseudomonas aeruginosa biofilms, Infect Immun 5:2383 (1990). Certain bacterial strains may be able to confer resistance protecting the biofilm from host defense components that would otherwise bind to the surface of viable bacteria and fill them. See D. Grenier and M. Belanger, Protective effect of Porphyromonas gingivalis outer membrane vesicles against bactericidal activity of human serum, Infect Immun 59:3004 (1991).

Therefore, targeting specific pathogenic bacteria for eradication from the bacterial biofilm would be beneficial in creating a more favorable bacterial community in the periodontal pocket. Moreover, the eradication of periodontal pathogenic bacteria from a mature biofilm would cause a shift in the bacterial ecology to resist the incorporation of these pathogenic bacteria back into a newly stabilized non-pathogenic bacterial biofilm in the periodontal pocket.

The gingival crevice or periodontal pocket region is walled on one side by the tooth surface and on the other side by gingival tissues. Both the tooth surface and the epithelial lining of the gingiva are areas where bacterial biofilms can form.

When a periodontal pocket forms, bacterial biofilm can cling tenaciously to the tooth root surface and planktonic bacteria or clumps of biofilm bacteria can invade and reside in the dentinal tubules of the tooth root surface.

At the gingival wall, the bacteria biofilm and the host response to it alter the integrity and topography of the gingival crevice surface. The gingival epithelial lining becomes ulcerated and invaginated with disease progression as it becomes a periodontal pocket. Thus, the bacterial biofilm can affix to an irregular epithelial surface and planktonic bacteria or clumps of biofilm bacteria can invade the epithelial lining through interstitial spaces. They can even invade individual epithelial cells. Thus, the infection is tenacious, persistent and difficult to eradicate.

There remains a need for delivery of selectively targeted antimicrobials at localized periodontal treatment sites.

### SUMMARY OF THE INVENTION

The present invention relates to the use of time release of selectively targeted antimicrobials from a carrier agent delivered into the periodontal pocket. The selectively targeted antimicrobials are specific toward one or more species of periodontal pathogens. The carrier agents allows for ease of delivery to the gingival crevice or periodontal pocket, retention at the localized site and sustained release of the selectively targeted antimicrobials.

In one embodiment of the present invention, selectively targeted antimicrobials specific against a singular bacterial species is delivered, with a carrier agent to a localized periodontal treatment site via syringe or other dental instrument. In another embodiment of the present invention, the selectively targeted antimicrobials are specific against a group of bacterial species. In yet another embodiment of the present invention, a group of selectively targeted antimicrobials are specific against a singular bacterial species. In still another embodiment of the present invention, a group of selectively targeted antimicrobials are specific against a group of bacterial species.

In one embodiment, the selectively targeted antimicrobial is a bactericidal peptide. In another embodiment, more than one bactericidal peptide are combined in a single molecule. In yet another embodiment, the bactericidal peptide is combined with a flexible peptide linker between each antimicrobial region forming a fusion peptide. In still another embodiment, a targeting moiety is fused to the molecule further enhancing the bactericidal efficacy.

In an embodiment of the present invention, the selectively targeted antimicrobial is able to penetrate the bacterial biofilm to successfully eliminate the targeted bacteria from the biofilm, favorably altering the biofilm of the gingival crevice or periodontal pocket to a new, stable and non-pathogenic biofilm. In another embodiment of the present invention, the carrier agent enhances penetration of the selectively targeted antimicrobial into the bacterial biofilm of the gingival crevice or periodontal pocket.

In an embodiment of the present invention, the carrier agent is a biocompatible, syringeable liquid or gel.

In another embodiment of the present invention, the selectively targeted antimicrobials are mixed into the carrier agent. In yet another embodiment of the present invention, the selectively targeted antimicrobials and carrier agent are in the form of a complex. In an embodiment of the complex, the antimicrobial can be caged within the molecular structure of the carrier agent. In another embodiment of the present invention, the selectively targeted antimicrobials are covalently bound to the carrier agent.

In an embodiment, the carrier agent adheres to the tooth surface. In yet another embodiment, the carrier agent adheres to and penetrates the bacterial biofilm. In still another embodiment, the carrier agent penetrates and deposits into the gingival tissues. These various embodiments, along with the viscosity of the carrier agent, allow for retention and sustained release of the selectively targeted antimicrobials for maximum efficacy in the gingival crevice or periodontal pocket localized periodontal treatment sites.

### BRIEF DESCRIPTION OF THE DRAWINGS

In the drawings, in which like reference numerals and letters indicate corresponding parts throughout the several views:

FIGS. 1A through 1C are diagrammatic views illustrating the human periodontal anatomy, including an illustration of the healthy human periodontium in FIG. 1A, an illustration of the effects of gingivitis in FIG. 1B, and an illustration of the effects of periodontitis in FIG. 1C;

FIG. 2 is a diagrammatic view of dental plaque biofilm in a periodontal pocket.

FIG. 3 is a partial diagrammatic view illustrating the placement of selectively targeted antimicrobials with carrier agent into a periodontal pocket via dental syringe.

### DETAILED DESCRIPTION OF THE INVENTION

Accordingly, the present invention provides methods and compositions for treating or reducing the incidence of periodontal disease. Specifically, in a first aspect, the present invention provides a method of treating or reducing the incidence of periodontal disease comprising combining at least one selectively targeted antimicrobial agent with at least one carrier agent to form a therapeutic treatment composition and applying the therapeutic treatment composition to the gingival crevice or periodontal pocket of a human or animal via dental syringe or other appropriate dental instrument.

In a second aspect, the selectively targeted antimicrobial agents are bacterial peptides with a targeting peptide moiety and a flexible peptide linker forming a fusion peptide to enhance specific bacterial species targeting and bactericidal efficacy. These selectively targeted antimicrobial peptides are able to penetrate the gingival crevice or periodontal pocket biofilms due to a relatively small molecular size of 20-50 amino acid sequences. The selectively targeted antimicrobial agents can also be designed with a lipophilic end to further facilitate bacterial biofilm penetration.

In a third aspect, the combination of the carrier agent with the selectively targeted antimicrobial agents allows for substantively or retention and sustained release from the gingival crevice or periodontal pocket by adhesion to the tooth surface, the gingival soft tissue wall and/or the bacterial biofilm. Further, the carrier agent can be chosen to penetrate the gingival crevice or periodontal pocket soft tissues effecting deposition and retention of the selectively targeted antimicrobial agents in the gingival tissues.

In a fourth aspect, the selectively targeted antimicrobials are mixed into the carrier agent, caged within the molecular structure of the carrier agent and/or covalently bound to the carrier agent to further effect sustained release at the intended periodontal treatment site.

In an embodiment, the present method can eliminate or reduce bacterial periodontal pathogens from the gingival crevice or periodontal pocket biofilms to levels commensurate with periodontal health. Moreover, the new bacterial biofilms within the gingival crevice or periodontal pocket attain a newly stabilized bacterial ecosystem which will resist the re-establishment of bacterial periodontal pathogens within these biofilms. Accordingly, the condition within the gingival crevice will shift from disease to health.

Periodontal bacteria that can be excluded from the bacterial biofilms include Porphyromonas gingivalis, Tannerella forsythia, Treponema denticola, Aggregatibacter actinomycetemcomitans, Peptostreptococcus micros, Prevotella intermedia, Prevotella nigrescens, Camptocytophaga rectus, Eikenella corrodens, Fusobacterium nucleatum, various Actinomyces species, Veillonella, spirochetes, and black-pigmented bacteroides.

Selective targeting of key periodontal pathogens within the periodontal bacterial biofilms is a new approach treating or reducing the incidence of periodontal disease. Previous approaches involved the mechanical removal of bacteria from tooth and tooth root surfaces with the more recent additions of either killing the bacterial biofilm, disrupting the existing biofilm or inhibiting new biofilm formation. Applicant's U.S. Pat. No. 4,685,883 deals with killing the periodontal bacterial biofilms and U.S. Pat. Nos. 6,576,226 and 6,726,898 deal with inhibiting or disrupting the periodontal bacterial biofilms.

The present invention utilizes the novel approach of selectively killing only the periodontal pathogenic species within the periodontal bacterial biofilms and allowing the non-pathogenic bacteria in the periodontal bacterial biofilms to survive and remain intact. Thus, the new periodontal bacterial biofilms reach a new ecosystem stability favoring health rather than causing disease and they also resist repopulation by the periodontal pathogens.

Pathogen selective molecules based on the fusion of a species specific targeting peptide domain linked to an antimicrobial peptide domain is an embodiment of the present invention.

The carrier agent needs certain chemical, physical and biologic properties to allow for optimal efficacy of the selectively targeted antimicrobial peptides for the treating or reducing the incidence of periodontal disease. It needs to be biocompatible with the human or animal subject undergoing treatment. It also needs to be compatible with the selectively targeted antimicrobial agents used. Further, a carrier agent is bioadhesive to the tooth root, the gingival soft tissues of the periodontal pocket wall and/or the periodontal biofilm. In addition, a carrier agent needs to reach the depths and contours of the periodontal pocket, yet resist evulsion by gingival crevicular fluid flow. Gingival crevicular fluid is a serum transudate which has a constant outward flow to flush from the gingival crevice or periodontal pocket bacteria and other irritants. The outward gingival crevicular fluid flow tends to evulse treatment compositions placed into the periodontal pocket back out into the oral cavity. The gingival crevicular fluid is totally exchanged in less than two minutes time.

### Antimicrobial Peptides

Antimicrobial proteins and peptides, such as selectively targeted antimicrobial peptides, that can be employed in the present invention include those listed in U.S. Patent Publication Nos. 2003/0143234, 2004/0137482, 2004/0052814, 2004/0137482, 2008/0170991 and 2008/0286210, each of which is incorporated herein by reference. Such antimicrobial peptides include histatin, protegrin, and novispirin. In an embodiment, the antimicrobial peptide is or includes a histatin derivative, histatin 5 and Dhvar1.

Histatin peptides and the derivatives thereof are a class of anti-microbial peptides, which have antifungal and antibacterial activities against a variety of organisms including Streptococcus mutans, which is believed to be the principal cause of dental caries (tooth decay) in humans. Histatin peptides and the derivatives thereof are very heterogeneous in amino acid sequence. However, they share some characteristics, such as a low molecular mass (1 to 5 kDa), a positively charged domain of 10 to 25 amino acids, and the tendency to form amphipathic structures. Generally, the histatin peptides and derivatives thereof have a mode of action that is characterized as binding and disruption of membranes, even though some studies indicate that these peptides also act intracellularly. Histatin peptides form a group of electrophoretically distinct histidine-rich polypeptides with microbicidal activity that are present in human parotid and submandibular gland secretions.

In one embodiment, the anti-microbial peptide moiety of the present invention contains one or more anti-microbial peptides from the class of histatin peptides and the derivatives thereof. For example, the anti-microbial peptide moiety of the present invention contains one or more derivatives of histatin including, without limitation, histatin 5 having an amino acid sequence as shown in SEQ ID NO:1. Histatin 5 is a 24-residue peptide from human saliva with antifungal properties. Histatin 5 inhibits coaggregation between Porphyromonas gingivalis and Streptococcus mitis, two microbes that play a role in periodontal disease. See Y. Murakami et al., “Inhibitory effects of human salivary histatins and lysozyme on coaggregation between Porphyromonas gingivalis and Streptococcus mitis,” Infect. Immun. 59:3284 (1991).

In another embodiment, histatin-5 is combined with a flexible peptide linker between each antimicrobial region and a targeting moiety to form a fusion peptide as shown in SEQ ID NO:2. The DNA sequence of the fusion peptide of SEQ ID NO:2 is shown in SEQ ID

NO:3. The fusion peptide of SEQ ID NO:2 allows specific binding to S. mutans as the targeting moiety is the variable region of the heavy chain derived from the SWLA3 monoclonal antibody. Commercially, the 24-amino acid peptide histatin-5 is available from AnaSpec, Inc., San Jose, Calif. (catalog #61001).

In another embodiment, the anti-microbial peptide moiety of the present invention contains one or more derivatives of histatin including, without limitation, Dhvar 1 having an amino acid sequence as shown in SEQ ID NO:4. Dhvar 1 is a 14-residue cationic antimicrobial peptide and derivative of the 14 C-terminal amino acids of histatin-5. Dhvar 1 has a high propensity to form an α-helix. In another embodiment, Dhvar 1 is combined with a flexible peptide linker between each antimicrobial region and a targeting moiety to form a fusion peptide as shown in SEQ ID NO:5. The DNA sequence of the fusion peptide of SEQ ID NO:4 is shown in SEQ ID NO:6. The fusion peptide of SEQ ID NO:5 allows specific binding to S. mutans as the targeting moiety is the variable region of the heavy chain derived from the SWLA3 monoclonal antibody. Dhvar-1 can be obtained via solid-phase peptide synthesis.

In another embodiment, the anti-microbial peptide moiety of the present invention contains one or more anti-microbial peptides from a class of protegrins and the derivatives thereof. For example, the anti-microbial peptide moiety of the present invention contains protegrin PG-1 having an amino acid sequence as shown in SEQ ID NO:7. The protegrin peptides have been shown to have anti-microbal effects on S. mutans. Protegrin peptides are described in the U.S. Pat. Nos. 5,693,486, 5,708,145, 5,804,558, 5,994,306, and 6,159,936, all of which are incorporated herein by reference.

In yet another embodiment, the anti-microbial peptide moiety of the present invention contains one or more anti-microbial peptides from a class of novispirin and derivatives thereof. For example, the anti-microbial peptide moiety of the present invention can be or include novispirin G10 having an amino acid sequence as shown in SEQ ID NO:8.

In another embodiment, the antimicrobial peptide includes a targeting peptide which is covalently linked to a linker peptide via a peptide bond and an antimicrobial peptide which is covalently linked to the linker peptide via a peptide bond as shown in SEQ ID NO:9 (C16G2), SEQ ID NO:10 (M8G2), SEQ ID NO:11 (M8-33), SEQ ID NO:12 (1903 BD2-21), SEQ ID NO:13 C16 BD2.21. For each of SEQ ID NOs. 9-13, the targeting peptide is specific for S. mutans and efficacy has been demonstrated for reducing the biofilms formed by S. mutans, and other microbial pathogenic microbes.

In another embodiment, the antimicrobial peptides that are useful in the present invention are shown in SEQ ID NO:14 (FBα-20), SEQ ID NO:15 (FBα-21), SEQ ID NO:16 (FBRW-15), SEQ ID NO:17 (FBRW-16), SEQ ID NO:18 (FBRW-22), and SEQ ID NO:19 (FBRW-24). For each of SEQ ID NOs. 14-19, efficacy has been demonstrated for reducing the colony forming units of S. mutans in cell culture.

The anti-microbial peptides of the present invention can include one or more anti-microbial peptides, which can be the same or different anti-microbial peptides. The anti-microbial peptides of the present invention can also be modified, e.g., to enhance its anti-microbial effectiveness, its cell delivery, its compatibility with the rest of the composition structure, or the manipulation of the composition in production.

In one embodiment, the antimicrobial peptide according to the present invention comprises about 2 to about 100 amino acids, from about 5 to about 50, or from about 20 to about 40. In another embodiment, the antimicrobial peptide has the antimicrobial activity with a minimum inhibitory concentration (MIC) of no more than about 40 μM, no more than about 30 μM, no more than 20 μM, or no more than 10 μM.

The antimicrobial peptides according to the present invention can be produced by any suitable method known to one skilled in the art by itself or in combination with a targeting peptide and a linker peptide. For example, the antimicrobial peptides can be chemically synthesized via a synthesizer or recombinantly made using an expression system, e.g., a bacterial, yeast, or eukaryotic cell expression system. In the chemical synthesis, the antimicrobial peptide can be made by L-amino acid enantiomers or D-amino acid enantiomers. As noted in the alternative above, the antimicrobial peptides can be purchased from one or more vendors.

### Carrier Agents

Carrier agents can include polylactide, polyglycolide, polylactide-co-glycolide, polycaprolactone, cellulosic—based polymers, ethylene glycol polymers and its copolymers, oxyethylene polymers, polyvinyl alcohol, chitosan and hyaluronan and its copolymers.

In an aspect, the carrier agents include hydroxyethyl cellulose, hydroxypropyl cellulose, hydroxypropylmethyl cellulose, hydroxymethyl cellulose, polyvinyl alcohol, polyethylene glycol, polyethylene oxide, ethylene oxide—propylene oxide co-polymers, chitosan, hyaluronan and its copolymers, or combinations thereof.

In another aspect, the carrier agents include hyaluronan or hyaluronic acid and copolymers including salts of hyaluronic acid, esters of hyaluronic acid, cross-linked gels of hyaluronic acid, enzymatic derivatives of hyaluronic acid, chemically modified derivatives of hyaluronic acid or combinations thereof. As used herein, hyaluronic acid broadly refers to naturally occurring, microbial and synthetic derivatives of acidic polysaccharides of various molecular weights constituted by residues of D-glucuronic acid polysaccharides and N-acetyl-D-glucosamine.

In an aspect, the selectively targeted antimicrobial agents can be mixed with the carrier agents, molecularly caged within the carrier agents or covalently bound to the carrier agents or a combination of the above.

In an aspect, the carrier agent is able to penetrate the gingival tissues through the soft tissue wall of the gingival crevice or periodontal pocket to access invaginations or crypts containing bacterial biofilm along the convoluted soft tissue surface. In a further preferred aspect, the carrier agent increases the deposition and retention of the selectively targeted antimicrobial agents within the gingival tissues of the gingival crevice or periodontal pocket. The interstitial spaces of the gingival epithelium provide access for an appropriate carrier agent to penetrate the epithelial and gingival tissues. Also, an appropriate carrier agent may act to enhance the penetration of the gingival tissues via movement through the vasculature.

In an embodiment, hyaluronic acid and derivatives of hyaluronic acid have affinity to inflamed gingival tissues. Moreover, the integrity of the gingival crevice or periodontal pocket epithelium is changed by periodontal disease activity to increase the interstitial spaces and cause microulcerations or breaches in the epithelial lining favoring penetration deposition and retention of the carrier agent in the gingival tissues. The cell surface receptor proteins RHAMM and CD44 aid in this process.

It will be appreciated that the therapeutic treatment compositions of the present invention can occur in many forms depending upon their intended mode of delivery and that inactive substances such as diluents, solvents, fillers, flavorings, stabilizer or other ingredients used to facilitate the combination, handling, stability or other properties of the therapeutic treatment compositions may be incorporated. Specifically, the therapeutic treatment composition may occur in a solid form, a semi-solid form (e.g., gel or paste), a liquid form or combinations thereof.

### Embodiments of the Present Method and Composition

In an embodiment, the present invention includes a method of treating or reducing the incidence of periodontal disease. This method can include applying to the gingival crevice or periodontal pocket a composition comprising a selectively targeted antimicrobial peptide with a carrier agent. In the composition applied, the carrier agent can provide retention, tissue penetration, deposition and sustained release of the selectively targeted antimicrobial agent for reducing the population of specific bacterial species within a periodontal biofilm and associated tissues. In an embodiment, the carrier agent provides penetration and retention into the gingival crevice or periodontal pocket and associated tissues with sustained release of the selectively targeted antimicrobial agent to enhance the reduction in population of select bacteria within the gingival tissue and dentinal tubule tissue.

In an embodiment, the selectively targeted antimicrobial peptide comprises a peptide targeting moiety coupled to a peptide antimicrobial moiety. The peptide targeting moiety and the peptide antimicrobial moiety can be linked via a flexible peptide to form a fusion peptide.

In an embodiment, the selectively targeted antimicrobial agent and the carrier agent are in the form of an admixture, in the form of a complex, covalently coupled, or a combination thereof.

In an embodiment, the carrier agent is bioadhesive. Suitable bioadhesive carrier agents include a cellulose based polymer. Suitable cellulose based polymers include hydroxyethyl cellulose, hydroxypropyl cellulose, hydroxymethyl cellulose, or a mixture thereof. In an embodiment, the bioadhesive carrier agent includes polylactide, polyglycolide, polylactide-co-glycolide, polyethylene glycol, hyaluronan, hyaluronic acid, chitosan, or a mixture thereof. The bioadhesive carrier agent can include a copolymer comprising polyethylene glycol, hyaluronan, hyaluronic acid, chitosan, or a mixture thereof.

In an embodiment, the carrier agent penetrates periodontal tissues. Suitable penetrating carrier agents include hyaluronic acid, a hyaluronic acid derivative, chitosan, a chitosan derivative, or a mixture thereof In an embodiment, the penetrating carrier agent includes a salt of hyaluronic acid, an ester of hyaluronic acid, an enzymatic derivative of hyaluronic acid, a cross-linked gel of hyaluronic acid, a chemically modified derivative of hyaluronic acid, or a mixture thereof.

The present invention also includes a therapeutic composition, which can be effective for treating or reducing the incidence of periodontal disease. The composition can include a selectively targeted antimicrobial agent and a carrier agent. The carrier agent can be effective to deliver or retain the composition at a secondary reservoir site of periodontal pathogens within the oral cavity. Such reservoir sites include supragingival plaque, buccal mucosa, tongue, a peritonsular region, or saliva. The therapeutic composition can be in the form of a lozenge, pastille, mouthrinse, mouth spray, aerosol, toothpaste, or a swab or toothbrush applied viscid liquid, gel or paste.

### Illustrated Embodiments

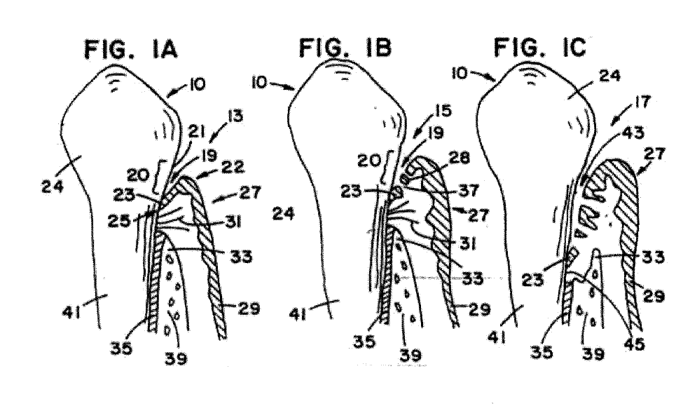

Referring now to FIGS. 1A through 1C, wherein there is diagrammatically illustrated a human periodontal anatomy 10, progressing from a healthy human periodontium 13 illustrated in FIG. 1A to a periodontium afflicted with periodontitis 17 illustrated in FIG. 1C.

Specifically, FIG. 1A illustrates a healthy human periodontium 13. Between the gingival margin 21 and the free gingiva 22 is the healthy gingival sulcus or crevice 19. The depth 20 of the gingival sulcus or crevice 19, from the gingival margin 21 to the attachment of the junctional epithelium 23, is approximately 1-3 millimeters. The junctional epithelium attaches to the tooth 24 at the cementoenamel junction (CEJ) 25. The gingival tissues 27, including the epithelium 29 and gingival fibers 31, are healthy and without inflammation. The alveolar bone crest 33 and periodontal ligament are undamaged.

FIG. 1B illustrates the human periodontium afflicted with gingivitis 15. The gingival tissues 27 show signs of inflammation and crevicular ulceration 37, resulting in white cell infiltration into the gingival sulcus or crevice 19. Furthermore, the ulcerations 37 in the crevicular epithelium 28 result in bleeding upon provocation, such as through brushing and flossing or mastication.

FIG. 1C illustrates the human periodontium afflicted with periodontitis 17. The gingival tissues 27 are inflamed. The alveolar bone crest 33 and periodontal ligament 35 have broken down due to both bacterial and host defense factors. The breakdown of the attachment of the alveolar bone 39 and periodontal ligament 35 to the tooth root 41 has resulted in the formation of a periodontal pocket lesion 43. In addition, apical proliferation of the junctional epithelium 23 is noted along the root surface 45. A chronic white cell infiltrate in the periodontal pocket lesion 43 is persistent. If left untreated, the continual loss of alveolar bone tissue 39 would result in the loss of the tooth 24.

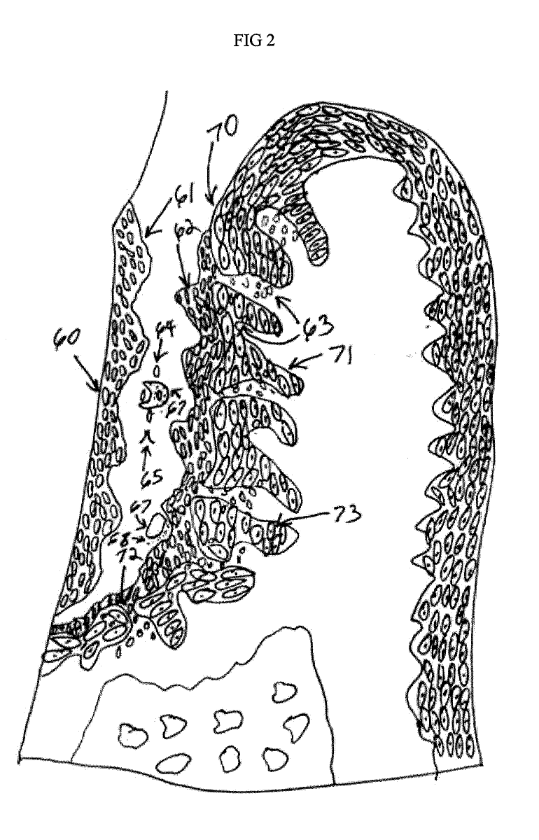

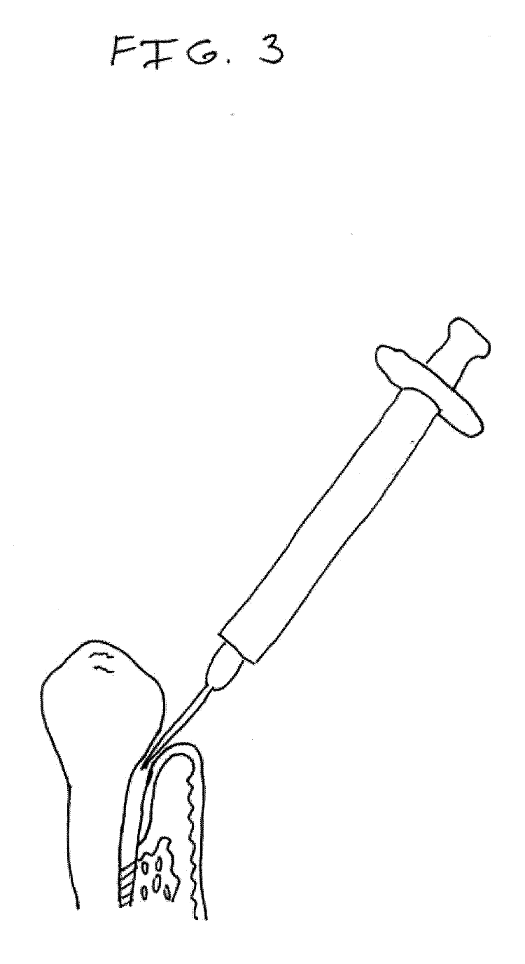

FIG. 2 illustrates a dental plaque biofilm 61 on a tooth surface 60 and a distinctly different dental plaque biofilm 62 on the periodontal pocketing soft tissue wall 70. Both biofilms co-exist simultaneously. Planktonic bacteria 64 can reside between the biofilms. Bacteria or bacterial clusters 63 can invade the interstitial spaces of the diseased periodontal pocket soft tissue epithelial lining 71 via breaches in the epithelial lining 72 or via widened interstitial spaces induced by inflammation 73. Planktonic bacteria 64 can be adequately cleared by antibodies 65 and neutrophils 67. Neutrophils 67 are also attracted to the dental plaque biofilms 61 and 62. Phagocytosis is frustrated at these biofilms yet phagocytic enzymes 68 are released which contribute to damage host tissue. The damage of host tissue elicited by both pathogenic bacteria within the biofilms and host phagocytic enzymes provide a source of nutrients for the periodontal pathogens within the biofilms to flourish. FIG. 3 illustrates placement of selectively targeted antimicrobial agents with carrier agent into the periodontal pocket via dental syringe.

### Additional Embodiments

Secondary reservoirs of periodontal pathogens exist in the oral cavity. These reservoirs include, but are not limited to, supragingival plaque, buccal mucosa, tongue, peritonsular regions and saliva. The periodontal pathogens not only reside in the interstitial or intercellular regions but are found within individual host epithelial cells as well. Further, bacterial masses inside oral mucosal cells share the polymicrobial nature of tooth surface biofilm. There is strong evidence to suggest that the shedding buccal epithelial surface is capable of supporting surprisingly large bacterial populations. Moreover, those populations may include relatively greater numbers of some pathogenic species than are found on supragingival tooth surfaces. This seems consistent with the hypothesis that buccal cells may provide a reservoir for subgingival recolonization. See J. D. Rudney, et al, Actinobacillus actinomycetemcomitans, Porphyromonas gingivalis, and Tannerella forsythensis are components of a polymicrobial intracellular flora within human buccal cells, J Dent Res 84:59 (2005).

Therefore, in another aspect of this invention the selectively targeted antimicrobial agents are transported, via the carrier agent, inside the oral mucosal tissues with retention and deposition for sustained release targeting periodontal pathogens in interstitial or intercellular regions. Further, in another aspect, the carrier agent facilitates transport of the selectively targeted antimicrobial agents into individual host cells targeting periodontal pathogens residing there. In an embodiment, hyaluronic acid and derivates of hyaluronic acid have affinity for both gingival and oral mucosal tissues and facilitate deposition and retention of selectively targeted antimicrobials within these tissues.

Such embodiments include, but are not limited to, mouthrinses, mouth sprays, aerosols, tooth pastes, swab or toothbrush applied viscid liquids, gels or pastes, lozenges and pastilles. Again, it will be appreciated that therapeutic treatment compositions of the present invention can occur in many forms depending upon their intended mode of delivery and that inactive substances such as diluents, solvents, fillers, flavorings, stabilizers or other ingredients used to facilitate the combination, handling, stability or other properties of the therapeutic treatment compositions may be incorporated.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

I claim:

1. A method of treating or reducing the incidence of periodontal disease comprising:
applying to a gingival crevice or periodontal pocket a composition comprising a selectively targeted antimicrobial peptide with a carrier agent;
wherein the carrier agent provides retention, tissue penetration, deposition and sustained release of the selectively targeted antimicrobial agent for reducing a population of specific bacterial species within a periodontal biofilm and associated tissues.

2. The method of claim 1, wherein the selectively targeted antimicrobial peptide comprises a peptide targeting moiety coupled to a peptide antimicrobial moiety.

3. The method of claim 2, wherein the peptide targeting moiety and the peptide antimicrobial moiety are linked via a flexible peptide to form a fusion peptide.

4. The method of claim 1, wherein the carrier agent provides penetration and retention into the gingival crevice or periodontal pocket and associated tissues with sustained release of the selectively targeted antimicrobial agent to enhance the reduction in population of select bacteria within the gingival tissue and dentinal tubule tissue.

5. The method of claim 1, wherein the selectively targeted antimicrobial agent and the carrier agent are: in the form of an admixture, in the form of a complex, covalently coupled, or a combination thereof.

6. The method of claim 1, wherein the carrier agent is bioadhesive.

7. The method of claim 6, wherein the carrier agent comprises a cellulose based polymer.

8. The method of claim 7, wherein the cellulose based polymer comprises hydroxyethyl cellulose, hydroxypropyl cellulose, hydroxymethyl cellulose, or a mixture thereof.

9. The method of claim 6, wherein the carrier agent comprises polylactide, polyglycolide, polylactide-co-glycolide, polyethylene glycol, hyaluronan, hyaluronic acid, chitosan, or a mixture thereof.

10. The method of claim 6, wherein the carrier agent comprises a copolymer comprising polyethylene glycol, hyaluronan, hyaluronic acid, chitosan, or a mixture thereof.

11. The method of claim 1, wherein the carrier agent penetrates periodontal tissues.

12. The method of claim 11, wherein the carrier agent comprises hyaluronic acid, a hyaluronic acid derivative, chitosan, a chitosan derivative, or a mixture thereof.

13. The method of claim 11, wherein the carrier agent comprises a salt of hyaluronic acid, an ester of hyaluronic acid, an enzymatic derivative of hyaluronic acid, a cross-linked gel of hyaluronic acid, a chemically modified derivative of hyaluronic acid, or a mixture thereof.

14. A therapeutic composition for treating or preventing periodontal disease comprising a selectively targeted antimicrobial agent and a carrier agent;
wherein the carrier agent is effective to deliver or retain the composition at a secondary reservoir site of periodontal pathogens within the oral cavity.

15. The therapeutic composition of claim 14, wherein the reservoir site comprises supragingival plaque, buccal mucosa, tongue, a peritonsular region, or saliva.

16. A therapeutic composition according to claim 14, wherein the therapeutic composition is in the form of a lozenge, pastille, mouthrinse, mouth spray, aerosol, toothpaste, or a swab or toothbrush applied viscid liquid, gel or paste.


SEQUENCE LISTING (19 sequences)

SEQ ID NO: 1 - protein, 24 aa, Artificial
  histatin 5 peptide
DSHAKRHHGYKRKFHEKHHSHRGY

SEQ ID NO: 2 - protein, 165 aa, Artificial
  histatin 5 fusion protein
DSHAKRHHGYKRKFHEKHHSHRGYSGGGGSGGGGSGGGGSDVKLVESGGGLVNPGGSLKLSCAASGFTFSSYTMSWVRQTPEKRLEWVASISSGGTYTYYPDSVKGRFTISRDNAKNTLYLQMTSLKSEDTAMYYCSRDDGSYGSYYYAMDYWGQGTSVTVSSAS

SEQ ID NO: 3 - DNA, 563 bp, Artificial
  histatin 5 fusion DNA
ggatatccaccatggacttcgggttgagcttggttttccttgtccttactttaaaaggtgtccagtgtgatagccacgctaagcggcaccacggatataagcggaagttccacgagaagcaccactcgcacagaggatactctggtggcggtggctcgggcggaggtgggtcgggtggcggcggatccgacgtgaagcttgtggagtctgggggaggcttagtgaaccctggagggtccctgaaactctcctgtgcagcctctggattcactttcagtagctataccatgtcttgggttcgccagactccggagaagaggctggagtgggtcgcatccattagtagtggtggtacttacacctactatccagacagtgtgaagggccgattcaccatctccagagacaatgccaagaacaccctgtacctgcaaatgaccagtctgaagtctgaggacacagccatgtattactgttcaagagatgacggctcctacggctcctattactatgctatggactactggggtcaaggaacctcagtcaccgtctcttcagctagc

SEQ ID NO: 4 - protein, 14 aa, Artificial
  dhvar 1 peptide
KRLFKELKFSLRKY

SEQ ID NO: 5 - protein, 155 aa, Artificial
  Dhvar 1 fusion protein
KRLFKELKFSLRKYSGGGGSGGGGSGGGGSDVKLVESGGGLVNPGGSLKLSCAASGFTFSSYTMSWVRQTPEKRLEWVASISSGGTYTYYPDSVKGRFTISRDNAKNTLYLQMTSLKSEDTAMYYCSRDDGSYGSYYYAMDYWGQGTSVTVSSAS

SEQ ID NO: 6 - DNA, 533 bp, Artificial
  Dhvar 1 fusion DNA
ggatatccaccatggacttcgggttgagcttggttttccttgtccttactttaaaaggtgtccagtgtaagcggctgtttaaggagctcaagttcagcctgcgcaagtactctggtggcggtggctcgggcggaggtgggtcgggtggcggcggatccgacgtgaagcttgtggagtctgggggaggcttagtgaaccctggagggtccctgaaactctcctgtgcagcctctggattcactttcagtagctataccatgtcttgggttcgccagactccggagaagaggctggagtgggtcgcatccattagtagtggtggtacttacacctactatccagacagtgtgaagggccgattcaccatctccagagacaatgccaagaacaccctgtacctgcaaatgaccagtctgaagtctgaggacacagccatgtattactgttcaagagatgacggctcctacggctcctattactatgctatggactactggggtcaaggaacctcagtcaccgtctcttcagctagc

SEQ ID NO: 7 - protein, 18 aa, Artificial
  protegrin PG-1 peptide
RGGRLCYCRRRFCVCVGR

SEQ ID NO: 8 - protein, 16 aa, Artificial
  novospirin G10
KNLRRIIRKGIHIIKK

SEQ ID NO: 9 - protein, 35 aa, Artificial
  selectively/specifically targeted antimicrobial peptide (STAMP) chimeric polypeptide C16G2
TFFRLFNRSFTQALGKGGGKNLRIIRKGIHIIKKY

SEQ ID NO: 10 - protein, 27 aa, Artificial
  selectively/specifically targeted antimicrobial peptide (STAMP) chimeric polypeptide M8G2
TFFRLFNRGGGKNLRIIRKGIHIIKKY

SEQ ID NO: 11 - protein, 23 aa, Artificial
  selectively/specifically targeted antimicrobial peptide (STAMP) chimeric polypeptide M8-33
TFFRLFNRSGGGFKKFWKWFRRF

SEQ ID NO: 12 - protein, 22 aa, Artificial
  selectively/specifically targeted antimicrobial peptide (STAMP) chimeric polypeptide 1903-BD2.21
NIFEYFLEGGGKLFKFLRKHLL

SEQ ID NO: 13 - protein, 30 aa, Artificial
  selectively/specifically targeted antimicrobial peptide (STAMP) chimeric polypeptide C16-BD2.21
TFFRLFNRSFTQALGKGGGKLFKFLRKHLL

SEQ ID NO: 14 - protein, 28 aa, Artificial
  FBalpha-20
RWRRLLKKLHHLLHGGGFKKFWKWFRRF

SEQ ID NO: 15 - protein, 28 aa, Artificial
  FBalpha-21
RWRRLLKKLHHLLHGGGIKQLLHFFQRF

SEQ ID NO: 16 - protein, 21 aa, Artificial
  FBRW-15
IKQLLHFFQRFGGGRWRWRWF

SEQ ID NO: 17 - protein, 21 aa, Artificial
  FBRW-16
LKRFLKWFKRFGGGRWRWRWF

SEQ ID NO: 18 - protein, 21 aa, Artificial
  FBRW-22
RWRWRWFGGGFKKFWKWFRRF

SEQ ID NO: 19 - protein, 21 aa, Artificial
  FBRW-24
RWRWRWFGGGLKRFLKWFKRF